(19) United States
(12) Patent Application Publication
(10) Pub. No.: US 2023/0181391 A1
(43) Pub. Date: Jun. 15, 2023

(54) ABSORBENT ARTICLE WITH IMPROVED STRUCTURAL FUNCTIONALITY COMMUNICATION

(71) Applicants: ONTEX BV, Buggenhout (BE); ONTEX GROUP NV, Erembodegem (BE)

(72) Inventors: Liliana Sanchez SERRANO, Puebla (MX); Denisse Torres GONZALEZ, Puebla (MX); Gabriela Galindo VEGA, Puebla (MX); María Cristina Romo GUTIERREZ, Puebla (MX); Karla Paulina Hernandez RAMOS, Coronango (MX)

(21) Appl. No.: 17/924,200

(22) PCT Filed: May 11, 2020
(86) PCT No.: PCT/EP2020/063008

§ 371 Date: Nov. 9, 2022

(51) Int. Cl. A61F 13/513; A61F 13/511

(52) U.S. Cl. CPC A61F 13/51394; A61F 13/51104; A61F 2013/51377

(57) ABSTRACT

The present invention relates to absorbent articles (1) and arrays (10) of absorbent articles such as sanitary napkins, incontinence pads, baby or adult diapers or pants, comprising a body facing surface (20), a garment facing surface (12) and an absorbent core (4) between the body facing surface (20) and the garment facing surface (12). The body facing surface (20) comprises at least one central region (2) comprising a first plurality of discrete elements (5a) viewable from the body facing surface (20) and two flanking regions (3a, 3b) comprising a second plurality of discrete elements (6a) viewable from the body facing surface (20). They are characterised in that the average perimeter of the elements from the central region is smaller than the average perimeter of the elements within the flanking regions. This may allow absorbent articles to have an improved structural functionality communication and an improved visual experience for the user.

### TECHNICAL FIELD

The invention pertains to the technical field of absorbent hygiene products. In particular, the present invention relates to absorbent articles such as sanitary napkins, panty liners, incontinence pads, baby diapers, baby pants, adult pants or the like, having an improved structural functionality communication and an improved visual experience for the user.

### BACKGROUND

Disposable absorbent articles such as sanitary napkins, panty liners, incontinence pads, baby diapers, baby pants, adult pants or the like, are employed to absorb and retain the body fluids from the user. Said articles consist essentially of a body facing surface; a garment facing surface and an absorbent core between the body facing surface and the garment facing surface. Each element that is part of the body facing surface of an absorbent article structure accomplishes a specific function, however sometimes the function of each element and the differences between elements is not clearly communicated to the user and/or customer.

Nowadays, the absorbent articles have ornamental features that intend to give an aesthetical appearance and improve the user's experience, this is commonly found in feminine hygiene absorbent articles like sanitary napkins, pantiliners and incontinence pad. Examples of such feminine hygiene absorbent articles comprising ornamental features, particularly on the body facing surface, have been widely disclosed, such as: USD641867, USD674483, USD816214, USD600803, USD630316 and USD664641.

While the ornamental features do improve the user's experience due to the aesthetical improvements, they fail to particularly point out to the user and/or to the consumer the specific different function of each element that is part of the body facing surface of such absorbent articles.

On the other hand, some attempts have been done to clearly differentiate a component of the body facing surface from another component in the same body facing surface, this is usually done using different colors or patterns, for example the patent P&G, EP1842513, which discloses a first and a second visually discernible lateral zones disposed over a central fluid acquisition zone. Although said visually discernible lateral zones are clearly different from the acquisition zone, the visual difference fails to clearly communicate the functionality of the lateral zones and the central zones.

Furthermore, despite the fact that the prior art has made multiples attempts to provide an improved aesthetical appearance to the body facing surface of the absorbent articles, there is still a need to provide as well an improved structural functionality communication that helps the user to better understand the purpose of the elements within the body facing surface and to be more comfortable during use. Moreover, there is a need of an improved structural functionality communication that is aesthetically appealing to improve significatively the user's experience.

### SUMMARY OF THE INVENTION

In a first aspect, the present invention relates to an absorbent article (1) comprising a body facing surface (20), a garment facing surface (12) and an absorbent core (4) between the body facing surface (20) and the garment facing surface (12), wherein the body facing surface (20) comprises:

a) at least one central region (2), and
b) at least two flanking regions (3a,3b)

The at least one central region (2) comprises a first plurality of discrete elements (5a) that are viewable from the body facing surface (20). The at least two flanking regions (3a, 3b) comprise a second plurality of discrete elements (6a) that are viewable from the body facing surface (20). Said first plurality of discrete elements (5a) comprises at least two elements with a first average perimeter, and said second plurality of discrete elements (6a) comprises at least two elements with a second average perimeter; wherein the first average perimeter is smaller than the second average perimeter.

In an embodiment of the present invention, the first plurality of discrete elements (5a) has a first maximum visual density and the second plurality of discrete elements (6a) has a second maximum visual density, wherein the first visual density may be higher than the second visual density.

In another embodiment of the present invention, the first plurality of discrete elements may comprises at least one element that is different from at least one element of the second plurality of discrete elements in at least one characteristic. In a further embodiment of the present invention, at least one of the first and second plurality of discrete elements may comprise at least one element that is different from at least a second element of said respective same plurality of discrete elements in at least one characteristic. In an embodiment of the present invention, the first plurality of discrete elements comprises a first number of different elements and the second plurality of discrete elements comprises a second number of different elements, wherein the first number of different elements may be equal or different to the second number of different elements. The characteristics that may differ within the discrete elements preferably are shape, size, orientation, L*, a* and b*.

In a second aspect, the present invention refers to an array of absorbent articles (10) comprising at least:

a first absorbent article (10a) comprising a body facing surface (20), a garment facing surface (12) and an absorbent core (4) between the body facing surface (20) and the garment facing surface (12); wherein the body facing surface (20) comprises:
a) at least one central region (2), and
b) at least two flanking regions (3a,3b)
wherein the at least one central region (2) comprises a first plurality of discrete elements (5a) that are viewable from the body facing surface (20); wherein each of the at least two flanking regions (3a, 3b) comprises a second plurality of discrete elements (6a) that are viewable from the body facing surface (20); wherein the first plurality of discrete elements (5a) comprises at least two elements with a first average perimeter, wherein the second plurality of discrete elements (6a) comprises at least two elements with a second average perimeter; and wherein the first average perimeter is smaller than the second average perimeter;
a second absorbent article (10b) comprising a body facing surface (20) a garment facing surface (12); and an absorbent core (4) between the body facing surface (20) and the garment facing surface (12); wherein the body facing surface (20) comprises:
a) at least one central region (2), and
b) at least two flanking regions (3a,3b)
wherein the at least one central region (2) comprises a third plurality of discrete elements (5b) that are viewable from the body facing surface (20); wherein each of the at least two flanking regions (3a, 3b) comprises a fourth plurality of discrete elements (6b) that are viewable from the body facing surface (20); wherein the third plurality of discrete elements (5b) comprises at least two elements with a third average perimeter, wherein the fourth plurality of discrete elements (6b) comprises at least two elements with a fourth average perimeter; and wherein the third average perimeter is smaller than the fourth average perimeter.

In an embodiment, the first plurality of discrete elements (5a) has a first maximum visual density, the second plurality of discrete elements (5b) has a second maximum visual density, the third plurality of discrete elements (6b) has a third maximum visual density and the fourth plurality of discrete elements (5b) has a fourth maximum visual density wherein at least one of the following pair of maximum visual densities may be substantially the same:

a) First and third maximum visual densities
b) Second and fourth maximum visual densities

In another embodiment, the four maximum visual densities may be different from each other.

In an embodiment, the first plurality of discrete elements (5a) has a first number of different elements, the second plurality of discrete elements (5b) has a second number of different elements, the third plurality of discrete elements (6b) has a third number of different elements and the fourth plurality of discrete elements (5b) has a fourth number of different elements; wherein at least one of the following pair of the numbers of different elements may be identical:

a) First and third number of different elements
b) Second and fourth number of different elements

In another embodiment, the four numbers of different elements may be different from each other.

In an embodiment, at least one of the following pair of plurality of discrete elements may preferably comprise substantially the same discrete elements:

a) First and third plurality of discrete elements (5a, 5b)
b) Second and fourth plurality of discrete elements (6a, 6b)

In another embodiment, the four pluralities of discrete elements may comprise different elements from each other.

In both aspects of the present invention, the at least two flanking regions (3a, 3b) of each absorbent article comprise a layer of material; and the central region (2) may preferably comprise a different layer of material, wherein each layer of material of the at least two flanking regions (3a, 3b) may at least partially overlap the layer of material of the central region (2).

In both aspects of the present invention, the plurality of discrete elements found within the central region may form a diffraction pattern.

### BRIEF DESCRIPTION OF THE DRAWINGS

FIG. 1 is a top plan view of an exemplary feminine hygiene absorbent article according to the present invention

FIG. 2 is schematic cross-section of the exemplary feminine hygiene absorbent article of FIG. 1 taken along line C-C

FIG. 3 is a top plan view of an exemplary feminine hygiene absorbent article according to the present invention

FIG. 4 is an array of two exemplary feminine hygiene absorbent articles within one package according to an embodiment of the present invention

FIG. 5 is an array of two exemplary feminine hygiene absorbent articles within one package according to an embodiment of the present invention

### DETAILED DESCRIPTION OF THE INVENTION

Unless otherwise defined, all terms used in the present invention, including technical and scientific terms, have meaning as commonly understood by one ordinary skilled in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. For example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, contains” and are inclusive or open-ended terms that specifies the presence of what follows e.g. component and do not exclude or preclude the presence of additional, non-recited components, features, element, members, steps, known in the art or disclosed therein. Herein, “comprise” and include” mean that other elements and/or other steps which do not affect the end result can be added. Each of these terms encompasses the terms “consisting of” and “consisting essentially of”.

“Centerline” as used herein means an imaginary line that is equidistant from lateral surfaces of the element referred to, typically running through said element such to divide said element into two substantially equal halves.

The recitation of numerical ranges by endpoints includes all numbers and fractions subsumed within that range, as well as the recited endpoints.

The expression “absorbent article” or “hygiene absorbent articles” refers to articles which absorb and contain menses or body exudates, and is intended to include feminine hygiene absorbent articles such as pantiliners, sanitary napkins and incontinence pads, as well as baby diapers, adult diapers and other articles worn in the crotch region of a garment.

The expression “disposable” refers to articles which are intended to be discarded after single use, composed or otherwise disposed of in an environmentally compatible manner. This means that they are not intended to be laundered or otherwise restored or reused as an absorbent article.

The expression “feminine hygiene absorbent articles” refers to articles which are worn by females which are intended to absorb and contain various exudates which are discharged from the body (e.g., blood, menses, and urine).

The expression “discrete element” refers to an element that is distinct and is individually separate from other elements.

“Average perimeter” is the addition of the perimeter of each discrete element that is located within a certain region divided between the number of elements in said region.

“Standard deviation” is a measure of the amount of variation or dispersion occurring in a set of values, such as a set of perimeters of the discrete elements found in a plurality of discrete elements.

“Maximum visual density” or “MVD” is determined by the number of discrete elements present within a square with an area of 6.25 cm².

With respect to the discrete elements described herein, except otherwise specified, when discussing about “difference” or being “different”, it is meant that the elements in comparison differ in at least one of the following characteristics: shape, size, orientation, L*, a* and b* (according to the CIELAB color space defined by the International Commission on Illumination (CIE) in 1976). Whilst different, the discrete elements described herein, may be “coordinated” and still have at least one, preferably at least two or three, more preferably at least four, of said characteristics in common. For example, a same shape and colour (i.e. all same L* a* b*) but a different size and orientation; or same size, shape, orientation, a* and b*, but a different colour lightness (i.e. L*).

With respect to the discrete elements described herein, except otherwise specified, when discussing about “sameness” or being the “same”, it is meant that the elements in comparison have all of the following characteristics in common: shape, size, orientation, L*, a* and b* (according to the CIELAB color space defined by the International Commission on Illumination (CIE) in 1976).

“Diffraction pattern” means herein a pattern formed by discrete elements with the same shape but decreasing in size toward the edges of the pattern and/or wherein the space between each discrete element increases toward the edges of the pattern.

Absorbent articles suitable for use herein are preferably feminine hygiene absorbent articles such as sanitary napkins but are not limited to only these products.

### Absorbent Article (1)

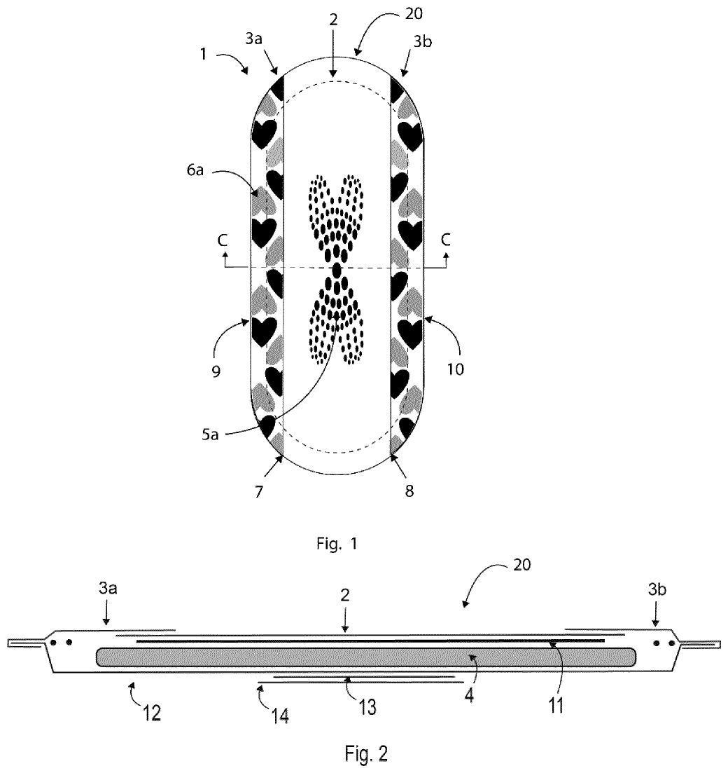

FIG. 1 shows a top plan view of an absorbent article (1), more particularly a feminine hygiene absorbent article which is one preferred embodiment of the present disclosure.

The absorbent article (1) comprises a body facing surface (20) and an opposite garment facing surface (12); and an absorbent core (4) between the body facing surface (20) and the garment facing surface (12). Said body facing surface (20) comprises at least one central region (2) and at least two flanking regions (3a, 3b).

FIG. 2 shows a schematic cross-section of the core of FIG. 1 taken along line C-C. In said figure the at least one central region (2) and the two flanking regions (3a, 3b) forming together the body facing surface (20) can be distinguished from each other.

### Body Facing Surface (20)

The absorbent article (1) comprises a body facing surface (20) that is in contact with the wearer skin during use and therefore is the first point of contact between the absorbent article and the body exudates. The body facing surface (20) comprises at least one central region (2) and at least two flanking regions (3a,3b).

Said at least one central region (2) and said at least two flanking regions (3a, 3b) may extend in the same direction, said direction being preferably the longitudinal direction.

### Central Region (2)

The body facing surface (20) of the absorbent articles (1) of the present invention has at least one central region (2) which may typically be aligned with the insult region of the user. The central region (2) is preferably differentiated from the flanking regions (3a, 3b) by visual discernible divisions (7,8).

As seen in FIG. 1, the central region (2) comprises a first plurality of discrete elements (5a) viewable from the body facing surface (20) of the absorbent article (1). The discrete elements may be printed through contact printing or non-contact printing, but they may as well be obtained through pigmentation and/or embossing process. Said discrete elements may be ornamental such as geometric figures, abstract figures, numbers, patterns and/or colors, but they may as well be elements that communicate or indicate a function such as words and symbols. Said first plurality of discrete elements (5a) comprise at least two elements and a first average perimeter. Said first plurality of discrete elements (5a) has as well a first maximum visual density.

The central region (2) may comprise a layer or a plurality of layers that are laminated between each other. Said layers may comprise any kind of nonwoven, for example spunbonded nonwoven, melt-blown nonwoven, SM nonwoven, SMS nonwoven, SMMS nonwoven, carded NW, film, foam or combinations of the same. The central layer could contain one or more types of natural or synthetic fibers. Said central region may comprise any kind of film layer as well, for example breathable film, non-breathable film, perforated film, elastomeric film or combinations of the same.

If desired, at least one layer of the central region (2) may be hydrophilic to promote the effective and rapid acquisition of body exudates. Said central region (2) may comprise embossments, apertures, ridges, channels, protrusions or any other mechanically produced features. It may comprise emollients, additives, surfactants or combinations thereof.

The central region (2) may comprise one or more acquisition and/or distribution layers (11), typically made of any nonwoven well known in the art such as air-through-bonded nonwovens that promote the acquisition and/or distribution of liquids such as body exudates and menses.

Therefore, when referring to the central region (2) comprising a first plurality of discrete elements (5a) viewable from the body facing surface (20) of the absorbent article (1), the discrete elements do not necessary need to be present on the outermost layer of the absorbent article (aimed to be in contact with user's skin), but they may be present on any other element of the central region (2), for example more internal layers like an acquisition and/or distribution layer or a core wrap layer, as soon as the elements are discernable for someone looking at the body facing surface (20) of the absorbent article (1).

### Flanking Regions (3a, 3b)

The body facing surface (20) of the absorbent article (1) of the present invention has at least two flanking regions (3a, 3b) which may preferably extend in longitudinal direction, preferably in the same direction as the central region (2). Said at least two flanking regions (3a, 3b) may work as antileakage barriers and/or to define the visual discernible divisions (7,8) between the flanking regions (3a, 3b) and the central region (2). Said flanking regions may be defined by external edges (9,10) and internal edges (7, 8) that act as the same time as visual discernible divisions between the flanking regions and the central region.

A preferred embodiment of the present invention is shown in FIGS. 1 and 2. In this embodiment the at least two flanking regions (3a, 3b) of the absorbent article may consist of a layer of material that is different from the layer(s) of material in the central region (2). Said different layer of material of the at least two flanking regions (3a, 3b) may at least partially overlap the layer or layers of the central region (2).

As seen in FIG. 1, the flanking regions (3a, 3b) comprise a second plurality of discrete elements (6a) that are viewable from the body facing surface (20) of the absorbent article. The second plurality of discrete elements (6a) may be preferably printed through contact printing or non-contact printing, but they may as well be obtained through pigmentation and/or embossing process.

Said second plurality of discrete elements (6a) comprise at least two elements and a second average perimeter. Said second plurality of discrete elements (6a) has a second maximum visual density.

Said flanking regions (3a, 3b) may be any kind of nonwoven as for example spunbonded nonwoven, melt-blown nonwoven, SM non-woven, SMS nonwoven, SMMS nonwoven, carded NW, film, foam or combinations of the same. Said flanking regions may be hydrophobic to prevent the leakage of the body fluids and menses that could run toward that region.

### Absorbent Core (4)

The absorbent core (4) is positioned between the body facing surface (20) and the garment facing surface (12). The absorbent core (4) may comprise materials such as fluff pulp, super absorbent particles (SAP), absorbent foam materials, absorbent nonwoven materials or mixtures thereof. The absorbent core (4) may be wrapped with a tissue or nonwoven material as is known in the art. The core wrap typically is constituted by a single layer of material enveloping the absorbent material or by an upper and lower layer of nonwoven bonded together. The absorbent core (4) may have any shape known in the art, such as rectangular shape, hourglass shape or “T” shape.

### Garment Facing Surface (12)

The garment facing surface (12) is placed opposite to the body facing surface (20). Said garment facing surface (12) comprises typically one or more hydrophobic layers that prevent the body exudates to get in contact with the underwear and/or clothes of the wearer. The materials that are typically used for the garment facing surface (12) are polyethylene or polypropylene films alone or in combination with nonwoven layers. The garment facing surface (12) may be a plain white or colored material. In a further embodiment the garment facing surface (12) may have a plurality of discrete elements viewable from said garment facing surface (12).

Absorbent articles such as feminine hygiene articles may further comprise on the garment facing surface (12) one or more adhesive regions (13), preferably extending in longitudinal direction, that are covered with a release paper (14) before use that is later removed to be adhered to the underwear of the users. The feminine hygiene articles may comprise a pouch such as to envelope the absorbent article in a folded configuration prior to use.

### Average Perimeter

The average perimeter of the plurality of discrete elements found either in the central region(s) (2) or the flanking regions (3a, 3b), is the addition of the perimeter of each discrete element that is located within a certain region divided between the number of discrete elements in said region.

In the case of the central region (2), the first average perimeter may be obtained by adding the perimeter of each discrete element of the first plurality of discrete elements found within the central region (2) and dividing said addition result by the total number of discrete elements found within said central region (2).

For the flanking regions (3a, 3b), the second average perimeter may be obtained by adding the perimeter of each discrete element of the second plurality of discrete elements found within either of the flanking regions (3a, 3b) and dividing said addition result by the total number of discrete elements found within said flanking region (3a, 3b).

The first average perimeter is smaller than the second average perimeter. This difference in perimeters helps to distinguish the structural functionality of each of the regions within the body facing surface. The bigger the average perimeter, the closer it may be perceived by the consumer and user, which consequentially may communicate that the determined region will approach more to the wearer's skin during use and thereby work effectively as anti-leakage barrier. In contrast, the smaller the average perimeter, the farther it may be perceived by the consumer and user, which consequentially may communicate that the determined region has a greater capacity of absorbing and retaining body exudates within its structure, far away from the user's body.

The standard deviation of the perimeters of at least one of the first and second plurality of discrete elements (5a, 6a) may be equal to or smaller than the respective first or second average perimeter; preferably, it is smaller. A smaller standard deviation provides a more homogeneous data set of perimeter values, communicating that the discrete elements share a similar or the same plane in the space.

### Maximum Visual Density (MVD)

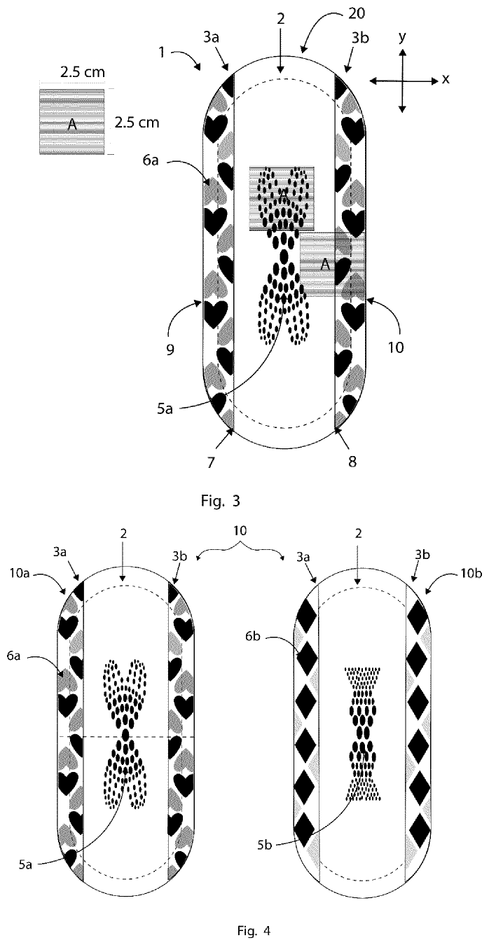

As seen in FIG. 3, the first and second maximum visual density (MVD) is determined by the number of the discrete elements of said first plurality or second plurality of discrete elements (5a, 6a), respectively, present within a square with an area “A” of 6.25 cm².

To obtain the first maximum visual density, said square with area “A” must be located where most of the discrete elements are located within the central region. The 1^{st} MVD is calculated by counting the number of discrete elements of the first plurality of discrete elements present within the area “A”.

To obtain the second maximum visual density, said square with area “A” must be located where most of the discrete elements are located within the flanking regions. The 2^{nd} MVD is calculated by counting the number of discrete elements of the second plurality of discrete elements present within the area “A”.

The first maximum visual density is higher than the second maximum visual density. This difference in densities may further help to distinguish the structural functionality of each of the regions within the body facing surface. A region with a higher maximum visual density that has a greater number of discrete elements may communicate a greater capacity of storing something, and in the present particular case, of absorbing a greater volume of body exudates. A region with a smaller visual density that has a smaller number of discrete elements may communicate lower capacity than the previously mentioned region. This contrast may communicate to the user the purpose of each of the elements that are part of the body facing surface.

As seen in FIG. 1, the first plurality of discrete elements (5a) comprises more than two discrete elements. Said elements may differ from each other in at least one of the following characteristics: shape, size, orientation, L*, a* and b* value. The second plurality of discrete elements (6a) as well comprises more than two discrete elements. Said elements may as well differ from each other in at least one of the following characteristics: shape, size, orientation, L*, a* and b* value.

When comparing the first and second plurality of discrete elements (5a, 6a), preferably at least one element of the first plurality of discrete elements (5a) is different from at least one element of the second plurality of discrete elements (6a) in at least one of the previously mentioned characteristics.

The first plurality of discrete elements (5a) may comprise a first number of different elements and the second plurality of discrete elements (6a) may comprise a second number of different elements, wherein the first number of different elements in the first plurality of discrete elements (5a) may or may not be different from the second number of different elements in the second plurality of discrete elements (6a).

As seen in FIGS. 1, in some embodiments of the present invention, the first plurality of discrete elements (5a) viewable from the body facing surface (20) of the absorbent article may form a diffraction pattern. Such patterns formed by discrete elements with the same shape but decreasing in size toward the edges of the central region, may impart an effect of greater volume capacity in said central region of the absorbent article.

FIG. 4 shows a further aspect of the present invention which is an array of absorbent articles (10) comprising at least a first absorbent article (10a) and a second absorbent article (10b). The body facing surface of the first absorbent article (10a) comprises at least one central region (2) and at least two flaking regions (3a, 3b). The central region (2) comprises a first plurality of discrete elements viewable from said body facing surface, said first plurality of discrete elements having a first average perimeter. The at least two flanking regions (3a, 3b) comprise a second plurality of discrete elements viewable as well from the body facing surface, said second plurality of discrete elements having a second average perimeter. The first average perimeter being smaller than the second average perimeter. The body facing surface of the second absorbent article (10b) comprises at least one central region (2) and at least two flaking regions (3a, 3b). The central region (2) comprises a third plurality of discrete elements viewable from said body facing surface, said third plurality of discrete elements having a third average perimeter. The at least two flanking regions (3a, 3b) comprise a fourth plurality of discrete elements viewable as well from the body facing surface, said fourth plurality of discrete elements having a fourth average perimeter. The third average perimeter being smaller than the fourth average perimeter.

In an embodiment of said aspect of the present invention, the at least two absorbent articles (10a, 10b) of the array (10) may each have at least two flanking regions (3a, 3b) with at least one different layer of material compared to the at least one layer of material of the central region (2).

The first, second, third and fourth plurality of discrete elements (5a, 6a, 5b, 6b) in the array of absorbent articles may show a respective first, second, third and fourth maximum visual densities, each visual density being determined by the number of discrete elements present in the area “A” on each determined region, as explained above. In an embodiment of the present invention, the maximum visual density of the first plurality of discrete elements (5a) may be substantially equal to the maximum visual density of the third plurality of discrete elements (5b); and/or the maximum visual density of the second plurality of discrete elements (6a) may be substantially equal to the maximum visual density of the fourth plurality of discrete elements (6b). In a further embodiment of the present invention, the first, second, third and fourth maximum visual densities may be all different from each other.

The first, second, third and fourth plurality of discrete elements (5a, 6a, 5b, 6b) in the array of absorbent articles may comprise a respective first, second, third and fourth number of different elements. In an embodiment of the present invention, the number of different elements of the first plurality of discrete elements (5a) may be equal to the number of different elements of the third plurality of discrete elements (5b); and/or the number of different elements in the second plurality of discrete elements (6a) may be equal to the number of different elements of the fourth plurality of discrete elements (6b). In a further embodiment of the present invention, the first, second, third and fourth number of different elements may be all different from each other.

In one embodiment of an array of absorbent articles, the first plurality of discrete elements (5a) may comprise substantially the same discrete elements found in the third plurality (5b) of discrete members; alternatively, or in addition, the second plurality of discrete elements (6a) may or may not comprise substantially the same discrete elements found in the fourth plurality of discrete elements (6b). In a further embodiment of the present invention, the array of absorbent articles may comprise different discrete elements in each of the plurality of discrete elements viewable from the body facing surface of the at least two absorbent articles.

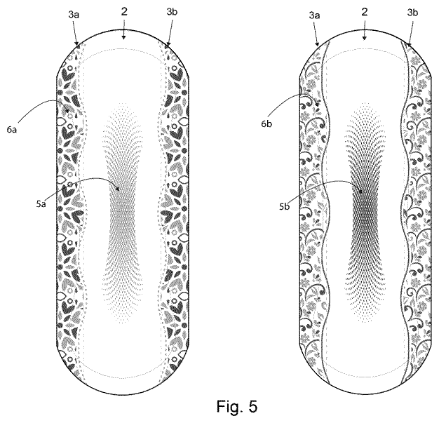

As seen in FIG. 5, both first and third plurality of discrete elements (5a, 5b) viewable from the body facing surface (20), may form a diffraction pattern in each of the at least two absorbent articles. In this latter example, the diffraction pattern is formed by discrete elements with the same shape wherein the space between each discrete element increases toward the edges of the central region (2).

It is supposed that the present invention is not restricted to any form of realization described previously and that some modifications can be added to the presented example of fabrication without reappraisal of the appended claims.

1. An absorbent article (1) comprising a body facing surface (20), a garment facing surface (12) and an absorbent core (4) between the body facing surface (20) and the garment facing surface (12); wherein the body facing surface (20) comprises:
a) at least one central region (2), and
b) at least two flanking regions (3a,3b)
wherein the at least one central region (2) comprises a first plurality of discrete elements (5a) that are viewable from the body facing surface (20); wherein each of the at least two flanking regions (3a, 3b) comprises a second plurality of discrete elements (6a) that are viewable from the body facing surface (20); wherein the first plurality of discrete elements (5a) comprises at least two elements with a first average perimeter, wherein the second plurality of discrete elements (6a) comprises at least two elements with a second average perimeter; and wherein the first average perimeter is smaller than the second average perimeter.

2. An absorbent article (1) according to claim 1, wherein each of the at least two flanking regions (3a, 3b) comprises a layer of material and the central region (2) comprises a different layer of material, wherein each layer of material of the at least two flanking regions (3a, 3b) is at least partially overlapping the layer of material of the central region (2).

3. An absorbent article (1) according to any of the preceding claims, wherein the first plurality of discrete elements (5a) has a first maximum visual density and the second plurality of discrete elements (6a) has a second maximum visual density, wherein the first visual density is higher than the second visual density.

4. An absorbent article (1) according to any of the preceding claims, wherein the first plurality of discrete elements (5a) comprises at least one element that is different from at least one element of the second plurality of discrete elements (6a) in at least one characteristic.

5. An absorbent article (1) according to any of the preceding claims, wherein at least one of the first and second plurality of discrete elements (5a, 6a) comprises at least one element that is different from at least a second element of said respective same plurality of discrete elements in at least one characteristic.

6. An absorbent article (1) according to any of the preceding claims, wherein the first plurality of discrete elements (5a) comprises a first number of different elements and the second plurality of discrete elements (6a) comprises a second number of different elements, wherein the first number of different elements is different from the second number of different elements.

7. An absorbent article (1) according to any of the previous claims, wherein the first plurality of discrete elements (5a) comprises a first number of different elements and the second plurality of discrete elements (6a) comprises a second number of different elements, wherein the first number of different elements (5a) is equal to the second number of different elements (6a)

8. An absorbent article (1) according to claims 4 to 7, wherein the elements differ in at least one of the following characteristics: shape, size, orientation, L*, a* and b*.

9. An absorbent article (1) according to any of the previous claims, wherein the first plurality of discrete elements (5a) forms a diffraction pattern.

10. An absorbent article (1) according to any of the previous claims, wherein the standard deviation of the perimeters of at least one of the first and second plurality of discrete elements is equal to or smaller than the respective first or second average perimeter.

11. An array of absorbent articles (10) comprising at least:
a first absorbent article (10a) comprising a body facing surface (20), a garment facing surface (12) and an absorbent core (4) between the body facing surface (20) and the garment facing surface (12); wherein the body facing surface (20) comprises:
a) at least one central region (2), and
b) at least two flanking regions (3a,3b)
wherein the at least one central region (2) comprises a first plurality of discrete elements (5a) that are viewable from the body facing surface (20); wherein each of the at least two flanking regions (3a, 3b) comprises a second plurality of discrete elements (6a) that are viewable from the body facing surface (20); wherein the first plurality of discrete elements (5a) comprises at least two elements with a first average perimeter, wherein the second plurality of discrete elements (6a) comprises at least two elements with a second average perimeter; and wherein the first average perimeter is smaller than the second average perimeter;
a second absorbent article (10b) comprising a body facing surface (20), a garment facing surface (12); and an absorbent core (4) between the body facing surface (20) and the garment facing surface (12); wherein the body facing surface (20) comprises:
a) at least one central region (2), and
b) at least two flanking regions (3a,3b)
wherein the at least one central region (2) comprises a third plurality of discrete elements (5b) that are viewable from the body facing surface (20); wherein each of the at least two flanking regions (3a, 3b) comprises a fourth plurality of discrete elements (6b) that are viewable from the body facing surface (20); wherein the third plurality of discrete elements (5b) comprises at least two elements with a third average perimeter, wherein the fourth plurality of discrete elements (6b) comprises at least two elements with a fourth average perimeter; and wherein the third average perimeter is smaller than the fourth average perimeter.

12. An array of absorbent articles (10) according to claim 11, wherein each of the at least two flanking regions (3a, 3b) of the first and the second absorbent articles (10a, 10b) comprises a layer of material; and the central region (2) comprises a different layer of material, wherein each layer of material of the at least two flanking regions (3a, 3b) is at least partially overlapping the layer of material of the central region (2).

13. An array of absorbent articles (10) according to any of the claims from 11 to 12, wherein the first plurality of discrete elements (5a) has a first maximum visual density, the second plurality of discrete elements (5b) has a second maximum visual density, the third plurality of discrete elements (6b) has a third maximum visual density and the fourth plurality of discrete elements (5b) has a fourth maximum visual density wherein at least one of the following pair of maximum visual densities are substantially the same:
a) First and third maximum visual densities
b) Second and fourth maximum visual densities

14. An array of absorbent articles (10) according to any of the claims from 11 to 12, wherein the first plurality of discrete elements (5a) has a first maximum visual density, the second plurality of discrete elements has a second maximum visual density (6a), the third plurality of discrete elements has a third maximum visual density (5b) and the fourth plurality of discrete elements (6b) has a fourth maximum visual density, wherein each of the first, second, third and fourth maximum visual densities are different from each other.

15. An array of absorbent articles (10) according to any of the claims from 11 to 14, wherein the first plurality of discrete elements (5a) has a first number of different elements, the second plurality of discrete elements (5b) has a second number of different elements, the third plurality of discrete elements (6b) has a third number of different elements and the fourth plurality of discrete elements (5b) has a fourth number of different elements; wherein at least one of the following pair of the numbers of different elements are identical:
a) First and third number of different elements
b) Second and fourth number of different elements

16. An array of absorbent articles (10) according to any of the claims from 11 to 14, wherein the first plurality of discrete elements (5a) has a first number of different elements, the second plurality of discrete elements (5b) has a second number of different elements, the third plurality of discrete elements (6b) has a third number of different elements and the fourth plurality of discrete elements (5b) has a fourth number of different elements; wherein each of the first, second, third and fourth number of different elements are different from each other.

17. An array of absorbent articles (10) according to any of the claims from 11 to 16, wherein at least one of the following pair of plurality of discrete elements comprise substantially the same discrete elements:
a) First and third plurality of discrete elements (5a, 5b)
b) Second and fourth plurality of discrete elements (6a, 6b)

18. An array of absorbent articles (10) according to any of the claims from 11 to 16, wherein the first, second, third and fourth plurality of discrete elements (5a) comprise different elements from each other.

19. An array of absorbent articles (10) according to claim 18, wherein the elements differ in at least one of the following characteristics: shape, size, orientation, L*, a* and b*.

20. An array of absorbent articles (10) according to any of the claims from 11 to 19, wherein the first and third plurality of discrete noticeable elements (5a, 6a) form a diffraction pattern.
